(19) United States
(12) Patent Application Publication
(10) Pub. No.: US 2020/0206131 A1
(43) Pub. Date: Jul. 2, 2020

(54) Philsring Oral Health Aid

(71) Applicant: Philip Ralph Belpasso, Fair Lawn, NJ (US)

(72) Inventor: Philip Ralph Belpasso, Fair Lawn, NJ (US)

(21) Appl. No.: 16/234,543

(22) Filed: Dec. 27, 2018

(51) Int. Cl. A61K 9/00; A61C 19/06; A61K 33/38; A61K 9/70

(52) U.S. Cl. CPC A61K 9/0053; A61K 9/7007; A61K 33/38; A61C 19/063

(57) ABSTRACT

This invention is health aid device which is contoured to the user's mouth so it can be placed over their lower teeth root area which is a key location to effect the salivary gland to the lymph nodes system which channels through the whole body. The device is made of antiseptic material including which can have mounted on it a Phillips head screw that can be made of a different metal that will form a bimetallic couple and as its shape is a vortex-ular cross. This will create an electrical potential between the two metals, allowing current to flow thusly creating a signature electro magnetic field thereby staging area for positive mindfulness via the power of the cross and based on the quantum field theory of physics then it be connected to the oral fixation craving and can be also be used as a psychotropic remedy as well.

In general, the present invention is directed an oral health aid which comprises of a made of silver or another antiseptic material that is inserted into the users mouth and placed over the lower gum where it will then aid the body's immune T-cells in their identifying of viruses and bacteria by their reaction to the silver's antiseptic presence which will then create a vaccine like condition.

In general, this wearing of the oral health aid will open up a channel of communication between the body's t-cells by this conscious wearing of the health aid, which is a physical communicative link to the immune system.

The result of this communication will be the enticement of the T-cells to use this newly acquired time toward doing a more critical monitoring of the body's cell DNA division sequence duplication process where if a identified virus may try to tamper with sequencing to make the cell into a host as seen in the provirus like Human Papillomavirus HPV incorporation into the host genome. Immuno-oncology and immunotherapy researchers state that there are T-regulatory cells that have been changed by the cancer cell to become enemies of their former t-cell allies.

Another object of this to improved method and means of the above character which comes in its unique ability of the oral health aid to be reshaped into a finger ring is mandated by the reality that when eating and sleeping there are times to better to wear the Philsring oral health Aid as a finger ring.

The foregoing and other aspects of the invention are carried out by this oral health aid can also be enhanced by having a real Phillips-head screw mounted on it as an ornament which has is a very mindful symbol being a cross which is the icon for Christ who healed through positive thinking which is coincidently another known title for the cross called to positive sign as seen on batteries that it will take to communicate which the immune system in the and the Phillips head is also the shape of a pyramid as seen from its side view. This internal Philsring oral health Aid being make of silver as a gum covering anti septic can be used entirely for that purpose however then it would be better to have the external finger ring be made of copper which is the external anti septic of choice.

### BRIEF DESCRIPTION OF THE DRAWINGS

Reference is now made to the present illustrated in FIGS. 1-7.

FIG. 1-2 is the Philsring Oral Health Aid in its primary shape as a gum covering immune system attention getting device. FIG. 3-5 are the first embodiment is a single finger ring that the oral aid can be reshaped into made by wrapping it around a finger with will result in its having an unusual over lapping, FIG. 6 is a second embodiment of a finger ring that the oral aid can be reshaped into is a spiral with two wrapping around a finger, FIG. 7 is a third embodiment of a two finger ring that the oral aid can be reshaped into so it can be worn around two.

FIG. 1 is the Philsring Healing Aid in its primary use as a gum-covering device with Phillip head screw mounted.

FIG. 2 is a view of the Philsring oral health aid in its shape which is designed to go into the mouth as an oral gum covering healing aid Phillip head screw mounted

FIG. 3 is an view of Philsring oral health Aid in its shape as a finger ring showing the over lapping and with a Phillip head screw mounted

FIG. 4 is a front view of FIG. 3

FIG. 5 is a side view of FIG. 3.

FIG. 6 is an view of Philsring oral health in the embodiment as a two fingered ring,

FIG. 7 is an view of Philsring oral health in the embodiment atwo fingered ring.

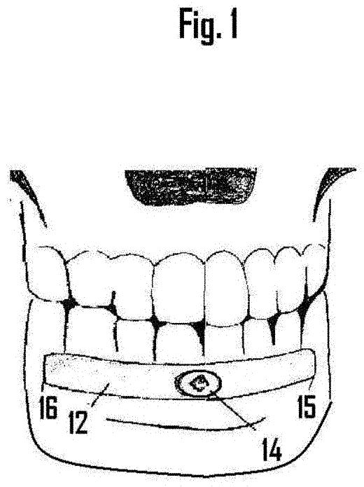

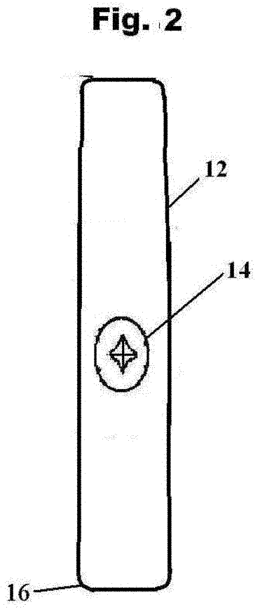

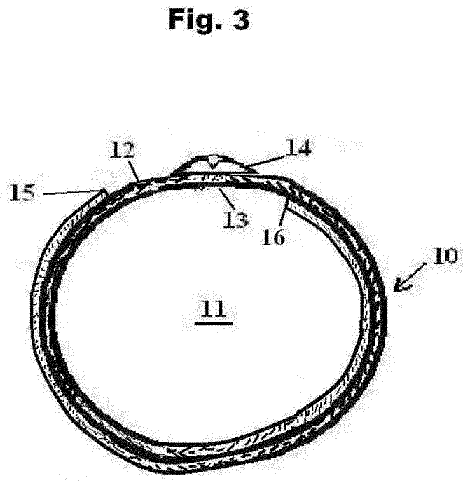

Which made from an antiseptic material such as silver has the ability to have its shape changed so it can function as either a oral health aid or a finger ring. In its rolled up shape it is a finger ring but can be unrolled with open ends 15,16 and made into a curved strip that can be inserted into the mouth and used as a health aid. The ring can have screwed into by way a hole 13 in its circumference so a real Phillips head screw that is short in length so it can be mounted into the hole 13 and not interfere with the internal aperture of 11 when the ring is in its rolled up shape, or irritate the gum when used in its oral health aid state. The Phillip's head screw is optional it function is to physiologically stimulate circulation by way of the cross on Phillip's head screw is representative of the positive attitude of the conscious mind over the bodies immune system which needs to be circulating to hard to reach places in the body such as outer skin and scare tissue.

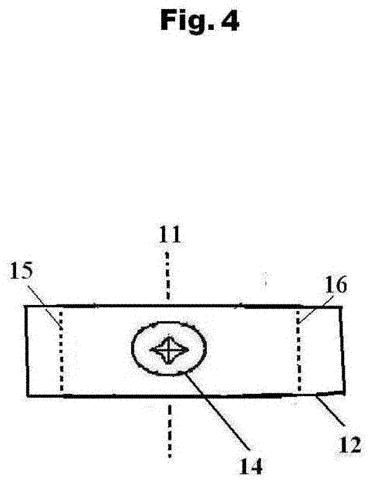

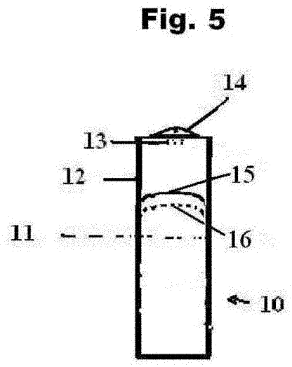

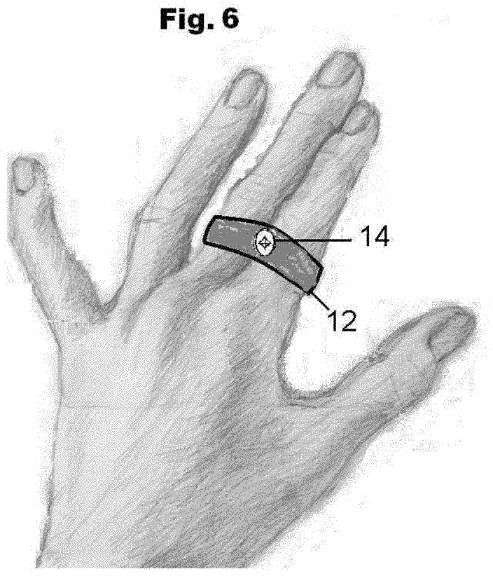

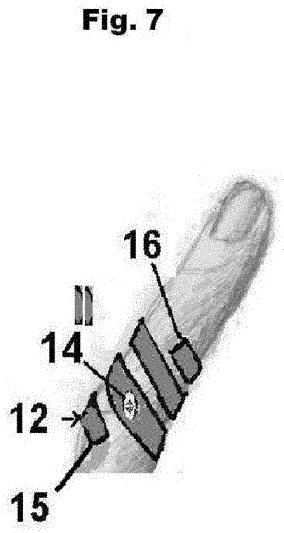

The antiseptic material such as silver which makes up section 12 when in use as a ring will contain a passage 11 which must be unobstructed by 13 the coupling of the Phillips head screw 14 which has its top head pointing outward from the passage 11 of the rolled up ring shape 10. FIG. 4 is a Philsring oral health aid, which has been reshaped from a finger ring by unrolling it so it can then be fitted in to the mouth of the user. FIG. 5 is the fitted oral health Aid showing its placement covering the lower jaw's gum area, which is similar if not identical to how it would be fitted to cover the upper jaw's gum area.

1: An oral health aid made of a nonflexible strip of antiseptic material which was shaped by copying a casting of the user's jaw shape including indentations to accommodate teeth roots being covered and having rounded edges on it circumference strip of antiseptic material which is a personalized length and gauge the shape of a person's jaw and inserted into their mouth and fitted over their gum teeth root area.

2: An oral health aid as described claim 1 however is made of a flexible strip of antiseptic material that can be uniquely reshaped into a finger ring which can accommodate the length which determined by the sizing of the jaw of the person which has fitted on it an ornament of a Philips-head screw.

3: An oral health aid finger ring as described claim 2 which is uniquely reshaped by being rolled up so its end will overlap to accommodate the length which is determined by the sizing of the jaw of the person.

4: An oral health aid finger ring as described claim 2 composed of a which will spiral around a finger length which is determined by the sizing of the jaw of the person.

5: An oral health aid finger ring as described claim 2 which is worn around two fingers to accommodate the length which is determined by the sizing of the jaw of the person.

6: An oral health aid as described claim 1 fitted on it an ornament of a Philips-head screw.

7: An oral health aid as described claim 2 which has fitted on it an ornament of a Philips-head screw.

8: An oral health aid as described claim 1 worn to satisfy the user's psychological oral fixation to have something in their mouth.

9: An oral health aid as described claim 2 worn to satisfy the user's psychological oral fixation to have something in their mouth.

10: An oral health aid as described claim 1 which can contain flavors, or drugs to satisfy the user.

11: An oral health aid as described claim 2 which can contain flavors, or drugs to satisfy the user.

12: An oral health aid as described claim 1 embedded with an ornament of a Phillips head screw composed of a differential material from the strip characterized so that the dissimilar materials of the strip and screw form a galvanic anode-cathode couple of electric potential inducing a current and electro-magnetic field to flow in the presence of saliva enhancing the direct antiseptic effect and the stimulation of immune response in the user.

13: An oral health aid as described claim 2 embedded with an ornament of a Phillips head screw composed of a differential material from the strip characterized so that the dissimilar materials of the strip and screw form a galvanic anode-cathode couple of electric potential inducing a current and electro-magnetic field to flow in the presence of saliva enhancing the direct antiseptic effect and the stimulation of immune response in the user.
